(19) United States
(12) Patent Application Publication
(10) Pub. No.: US 2019/0298852 A1
(43) Pub. Date: Oct. 3, 2019

(54) SELF-HEALING HYDROGEL AND USE THEREOF

(71) Applicant: INDUSTRY-UNIVERSITY COOPERATION FOUNDATION HANYANG UNIVERSITY, Seoul (KR)

(72) Inventors: Keun Yong LEE, Seoul (KR); Eun Seok KO, Seoul (KR)

(21) Appl. No.: 16/370,319

(22) Filed: Mar. 29, 2019

(30) Foreign Application Priority Data
Mar. 29, 2018 (KR) 10-2018-0036785

(51) Int. Cl. A61K 47/69; A61K 47/61; A61K 47/02; A61K 9/06; A61K 47/36

(52) U.S. Cl. CPC A61K 47/6903; A61K 47/61; A61K 45/06; A61K 9/06; A61K 47/36; A61K 47/02

(57) ABSTRACT

The present invention relates to a glycol chitosan-based hydrogel containing only natural polysaccharides without use of a toxic crosslinking agent. The hydrogel of the present invention exhibits a self-healing behavior under physiological conditions and in the presence of iron oxide nanoparticles, and thus can be useful for use as a delivery system for injection. In addition, in the hydrogel of the present invention, a delivery rate or amount of a substance carried within the hydrogel can be regulated using the magnetic field, which allows the hydrogel to be used for a variety of drug delivery and tissue engineering applications with on-demand release of gel payloads under external magnetic stimulation. The hydrogel can be effectively used as a delivery vehicle for a physiologically active substance such as a therapeutic agent.

### CROSS-REFERENCE TO RELATED APPLICATION

This application claims priority to and the benefit of Korean Patent Application No. 10-2018-0036785, filed Mar. 29, 2018, the disclosure of which is incorporated herein by reference in its entirety.

### BACKGROUND

### 1. Field of the Invention

The present invention relates to a self-healable hydrogel and a use thereof.

### 2. Discussion of Related Art

Among various types of biomaterials used in tissue engineering, hydrogels are very useful in various fields. Such hydrogels have been used as space fillers and bioactive molecule carriers, particularly cell delivery vehicles.

Hydrogels are polymeric networks fabricated by either covalent bonds or non-covalent bonds, which retain a large amount of water (Strandman, S. and X. X. Zhu (2016). “Self-Healing Supramolecular Hydrogels Based on Reversible Physical Interactions.” Gels 2(2):16). Compared to ceramic materials, hydrogels can be easily designed and fabricated from organic materials (Wang, Y., et al. (2017). “Recent development and biomedical applications of self-healing hydrogels.” Expert Opinion on Drug Delivery: 1-15). Thus, hydrogels have been widely used as biomaterials in many biomedical fields, including cell/drug delivery systems, scaffolds in tissue engineering, and the like. Recently, there has been a continuous need for improving the safety of hydrogels and enhancing the performance thereof.

In particular, at the time of injection of hydrogels, cracking occurs due to shear force, and cracking in hydrogel materials causes a problem that the mechanical properties thereof suddenly deteriorate (Wang, Y., et al. (2017). “Recent development and biomedical applications of self-healing hydrogels.” Expert Opinion on Drug Delivery: 1-15; and Taylor, D. L. and M. I. H. Panhuis (2016). “Self-Healing Hydrogels.” Advanced Materials 28(41):9060-9093). One way to extend the lifetime of hydrogels and improve the performance thereof is to make self-healing hydrogels, and self-healing properties of the hydrogels are very important to overcome limitations of conventional hydrogels.

Self-healing ability is defined as the property that enables a material to autonomously heal damage (Taylor, D. L. and M. I. H. Panhuis (2016). “Self-Healing Hydrogels.” Advanced Materials 28(41):9060-9093). A self-healing reagent typically leaks into a cracked region and reconstitutes a damaged portion in an autonomous self-healing material. Self-healing hydrogels are considered to be injectable by a syringe due to their properties of complete recovery to their original mechanical stiffness (Zhang, Y. L., et al. (2012). “A magnetic self-healing hydrogel.” Chemical Communications 48(74):9305-9307; and Lu, H. D., et al. (2012). “Injectable shear-thinning hydrogels engineered with a self-assembling Dock-and-Lock mechanism.” Biomaterials 33(7):2145-2153). Unlike previous hydrogels (which are not injectable), self-healing hydrogels can regenerate the damaged area (Deng, G. H., et al. (2012). “Dynamic Hydrogels with an Environmental Adaptive Self-Healing Ability and Dual Responsive Sol-Gel Transitions.” Acs Macro Letters 1(2):275-279; Yang, B., et al. (2012). “Facilely prepared inexpensive and biocompatible self-healing hydrogel: a new injectable cell therapy carrier.” Polymer Chemistry 3(12):3235-3238; and Li, Y. L., et al. (2012). “Injectable and biodegradable hydrogels: gelation, biodegradation and biomedical applications.” Chemical Society Reviews 41(6):2193-2221). Self-healing hydrogels can be classified into two groups based on dynamic covalent reactions and non-covalent reactions (Taylor, D. L. and M. I. H. Panhuis (2016). “Self-Healing Hydrogels.” Advanced Materials 28(41):9060-9093). Covalent reactions in self-healing hydrogels usually require the reapplication of the conditions used for polymerization or the application of an external stimulus such as pH and a magnetic field (Strandman, S. and X. X. Zhu (2016). “Self-Healing Supramolecular Hydrogels Based on Reversible Physical Interactions.” Gels 2(2):16); and Wang, Y, et al. (2017). “Recent development and biomedical applications of self-healing hydrogels.” Expert Opinion on Drug Delivery: 1-15).

There have been many reports on self-healing materials over the last 10 years (Hager, M. D., et al. (2010). “Self-Healing Materials.” Advanced Materials 22(47):5424-5430). Several self-healing hydrogels have been developed and evaluated for various applications (White, S. R., et al. (2001). “Autonomic healing of polymer composites.” Nature 409(6822):794-797). However, these hydrogels are not highly effective and have few types, which causes such hydrogels to be industrially used in a limited manner. Therefore, there is still a need for the development of hydrogels which have a self-healing ability and of which the self-healing ability can be regulated, in term of the development of hydrogels which can replace drug delivery vehicles or medical tissue engineering.

Among many types of biomaterials, glycol chitosan and hyaluronate are base materials for gel preparation. Chitosan is a linear polysaccharide of (1-4)-linked D-glucosamine and N-acetyl D-glucosamine residues (FIG. 2(b)) (Martin, L., et al. (2003). “Sustained buccal delivery of the hydrophobic drug denbufylline using physically cross-linked palmitoyl glycol chitosan hydrogels.” European Journal of Pharmaceutics and Biopharmaceutics 55(1):35-45; and Ravi Kumar, M. N. V. (2000). “A review of chitin and chitosan applications.” Reactive and Functional Polymers 46(1):1-27). This polycationic biopolymer is usually obtained by deacetylation of chitin (Rinaudo, M. (2006). “Chitin and chitosan: Properties and applications.” Progress in Polymer Science 31(7):603-632). Chitosan has been investigated in many tissue engineering applications because it is structurally similar to naturally-occurring glycosaminoglycans (GAGs) and is degradable by enzymes in the body (Singh, A., et al. (2006). “External stimuli response on a novel chitosan hydrogel crosslinked with formaldehyde.” Bulletin of Materials Science 29(3):233-238. 16). Chitosan hydrogels are formed by various cross-linking methods involving dialdehydes such as glyoxal and in particular glutaraldehyde. Aldehyde groups form covalent imine bonds and chemically cross-link the chitosan backbone (Berger, J., et al. (2004). “Structure and interactions in covalently and ionically crosslinked chitosan hydrogels for biomedical applications.” European Journal of Pharmaceutics and Biopharmaceutics 57(1):19-34).

Hyaluronate (HA) is a naturally occurring linear polysaccharide comprised of β-1,4-linked D-glucuronic acid (FIG. 2a). HA is one of the major GAGs and is found in mammalian tissue (Luo, Y., et al. (2000). “Cross-linked hyaluronic acid hydrogel films: new biomaterials for drug delivery.” Journal of Controlled Release 69(1):169-184). HA hydrogels are typically formed by covalent cross-linking with hydrazide modification, esterification, and annealing (Hennink, W. E. and C. F. van Nostrum (2002). “Novel crosslinking methods to design hydrogels.” Advanced Drug Delivery Reviews 54(1):13-36). In addition, HA has been combined with both alginate and collagen to form a hydrogel. HA is degradable by a hyaluronidase (Csoka, T., et al., Hyaluronidases in tissue invasion. Invasion & metastasis, 1996. 17(6): p. 297-311).

Various cross-linking methods for forming chitosan hydrogels and HA hydrogels have been recently reported (Berger, J., et al. (2004). “Structure and interactions in covalently and ionically crosslinked chitosan hydrogels for biomedical applications.” European Journal of Pharmaceutics and Biopharmaceutics 57(1):19-34; Luo, Y., et al. (2000). “Cross-linked hyaluronic acid hydrogel films: new biomaterials for drug delivery.” Journal of Controlled Release 69(1):169-184; and Hennink, W. E. and C. F. van Nostrum (2002). “Novel crosslinking methods to design hydrogels.” Advanced Drug Delivery Reviews 54(1):13-36). Among these cross-linking methods, covalent cross-linking methods exhibit good mechanical properties and can overcome the dissolution of hydrogels in physiological conditions (Hoffman, A. S. (2012). “Hydrogels for biomedical applications.” Advanced Drug Delivery Reviews 64(Supplement):18-23; and Hennink, W. E. and C. F. van Nostrum (2012). “Novel crosslinking methods to design hydrogels.” Advanced Drug Delivery Reviews 64(Supplement):223-236).

Generally, a crosslinking agent is required to form hydrogels. Various crosslinking methods for forming hyaluronate-based hydrogels have been reported. The carboxyl group and the hydroxyl group are the most widely used functional groups for forming a bond in hyaluronate hydrogels. Conventional crosslinking methods include hyaluronate esterification, hydrazide modification, self-crosslinking, crosslinking with polyfunctional epoxides, crosslinking with glutaraldehyde, carbodiimide chemistry, and the like. However, there are still limitations, such as a rapid degradation rate, low mechanical properties, harsh gelation conditions, and the formation of toxic by-products, in applying these methods to cell delivery.

### SUMMARY OF THE INVENTION

An object of the present invention is to provide non-toxic hydrogels in which the limitations of conventional self-healing hydrogels are overcome. In this respect, the present inventors have continuously studied to develop a hydrogel which is excellent in self-healing ability and of which the self-healing ability can be regulated under specific conditions, the hydrogel being prepared through a non-toxic chemical crosslinking method with no addition of a chemical crosslinking agent. As a result, the present inventors have developed a self-healing hydrogel which comprises all of glycol chitosan, oxidized hyaluronate, and iron oxide, and thus have completed the present invention. The hydrogel of the present invention is excellent in self-healing ability, and the self-healing properties thereof can be regulated under the conditions such as a magnetic field.

According to the above object, the present invention provides a hydrogel, comprising glycol chitosan and oxidized hyaluronate.

A degree of oxidation of the hyaluronate may be 10% to 80%.

The hydrogel may further comprise iron oxide.

The iron oxide may be contained in an amount greater than 1 wt % with respect to the total weight of the hydrogel.

The hydrogel may have a self-healing ability.

The hydrogel may contain the glycol chitosan and oxidized hyaluronate in the total amount of 1.0 wt % to 5.0 wt % with respect to the total weight of the hydrogel.

In addition, the present invention provides a physiologically active substance delivery vehicle having a self-healing ability, comprising the hydrogel.

The physiologically active substance may be selected from the group consisting of antibiotics, anti-cancer agents, analgesics, anti-inflammatory agents, anti-viral agents, anti-bacterial agents, proteins, peptides, nucleic acids, polysaccharides, lipids, carbohydrates, steroids, extracellular matrix materials, and cells.

The proteins may be selected from the group consisting of hormones, cytokines, enzymes, antibodies, growth factors, transcriptional regulatory factors, blood factors, vaccines, structural proteins, ligand proteins and receptors, cell surface antigens, and receptor antagonists.

The nucleic acids may be selected from the group consisting of oligonucleotides, DNA, RNA, and PNA.

In addition, the present invention provides a composition for three-dimensional bio-printing, comprising the hydrogel.

In addition, the present invention provides a method for preparing a self-healing hydrogel, comprising mixing glycol chitosan and oxidized hyaluronate.

The method for preparing a self-healing hydrogel may further comprise oxidizing hyaluronic acid to prepare oxidized hyaluronate.

The method for preparing a self-healing hydrogel may further comprise mixing iron oxide with the mixture of glycol chitosan and oxidized hyaluronate.

According to the present invention, it is possible to provide a hydrogel which is non-toxic, biocompatible, and retains a self-healing ability without using chemical crosslinking agents or dispersants. The hydrogel of the present invention has a self-healing ability in a magnetic field region, which makes it possible to regulate a delivery rate or amount of a substance carried within the hydrogel using the magnetic field. Thus, the hydrogel of the present invention can be effectively used as a delivery vehicle for a physiologically active substance such as a therapeutic agent.

### BRIEF DESCRIPTION OF THE DRAWINGS

The above and other objects, features, and advantages of the present invention will become more apparent to those of ordinary skill in the art by describing in detail exemplary embodiments thereof with reference to the attached drawings, in which:

FIG. 1 illustrates a schematic view which describes a magnetically-driven self-healing system;

FIG. 2A illustrates chemical structures of (a) oxidized hyaluronate.

FIG. 2B illustrates chemical structures of glycol chitosan;

FIG. 3 illustrates a schematic view which describes the gelation behavior between oxidized hyaluronate and glycol chitosan;

FIG. 4 illustrates results obtained by identifying FT-IR spectra of HA, OHA, and OHA/GC hydrogels, prepared according to an embodiment of the present invention;

FIG. 5A illustrates results obtained by identifying volume changes.

FIG. 5B illustrates results obtained by identifying swelling ratios of GC/OHA gels prepared with various mixing ratios in an embodiment of the present invention;

FIG. 6A illustrates results obtained by identifying changes in shear moduli of GC/OHA hydrogels (GC:OHA=5:1) prepared with various polymer concentrations, which were measured by a frequency sweep mode. FIG. 6B illustrates results obtained by identifying changes in storage shear moduli of GC/OHA hydrogels ([polymer]=3 wt %) prepared with various mixing ratios of GC and OHA, in an embodiment of the present invention;

FIG. 7A illustrates a view for identifying the degradation of hydrogels, prepared according to an embodiment of the present invention, in an in vitro physiological environment, which shows results obtained by identifying changes in remaining weights (%). FIG. 7B illustrates a view for identifying the degradation of hydrogels, prepared according to an embodiment of the present invention, in an in vitro physiological environment, which shows results obtained by identifying changes in storage shear moduli of GC/OHA hydrogels;

FIG. 8 illustrates results obtained by identifying changes in storage shear moduli of OHA/GC/Fe₃O₄ hydrogels ([Fe₃O₄]=5 wt %), prepared according to an embodiment of the present invention, depending on polymer concentration;

FIG. 9 illustrates results obtained by identifying changes in storage shear moduli of hydrogels, prepared according to an embodiment of the present invention, depending on the concentration of iron oxide nanoparticles (Fe₃O₄);

FIG. 10 illustrates a view showing a schematic description of a self-healing process in a GC/OHA/Fe₃O₄ hydrogel, prepared according to an embodiment of the present invention;

FIG. 11 illustrates results obtained by identifying viscoelastic properties of GC/OHA/Fe₃O₄ hydrogels, prepared according to an embodiment of the present invention (amplitude sweep carried out at 0% to 500% strain);

FIG. 12A illustrates results obtained by identifying rheological measurements of the self-healing behavior of (a) GC/OHA hydrogels. FIG. 12B illustrates results obtained by identifying rheological measurements of the self-healing behavior of GC/OHA/Fe₃O₄ hydrogels, prepared according to an embodiment of the present invention;

FIG. 13A illustrates results obtained by identifying the self-healing efficiency of GC/OHA/Fe₃O₄ hydrogels which contain, at various concentrations, iron oxide nanoparticles. FIG. 13B illustrates results obtained by identifying the self-healing efficiency of GC/OHA/Fe₃O₄ hydrogels which contain, at various concentrations, a polymer, prepared according to an embodiment of the present invention, and;

FIG. 14A illustrates results obtained by fabricating self-healing a hydrogel disk (diameter=11 mm, thickness=3 mm) of GC/OHA/Fe₃O₄ hydrogels prepared according to an embodiment of the present invention. FIG. 14B illustrates results obtained by causing the disks to be assembled. FIG. 14C illustrates results obtained by applying the magnetic field and identifying that the disks were self-healed after 10 minutes;

FIG. 14D illustrates a graph obtained by identifying changes in physical properties, after repeated deformation, of magnetic hydrogels having a self-healing ability, prepared according to an embodiment of the present invention;

FIG. 15A illustrates results obtained by identifying changes in properties, after self-healing, of hydrogel disks prepared, at [GC]=1.5 wt %, [OHA]=0.3 wt %, [Fe₃O₄]=5 wt %. FIG. 15B illustrates results obtained by identifying changes in properties, after self-healing, of hydrogel disks prepared, at [GC]=2 wt %, [OHA]=0.4 wt %, [Fe₃O₄]=5 wt %, according to an embodiment of the present invention; and

FIG. 16 illustrates a graph obtained by identifying release characteristics of a drug (BSA) from magnetic hydrogels having a self-healing ability, prepared according to an embodiment of the present invention.

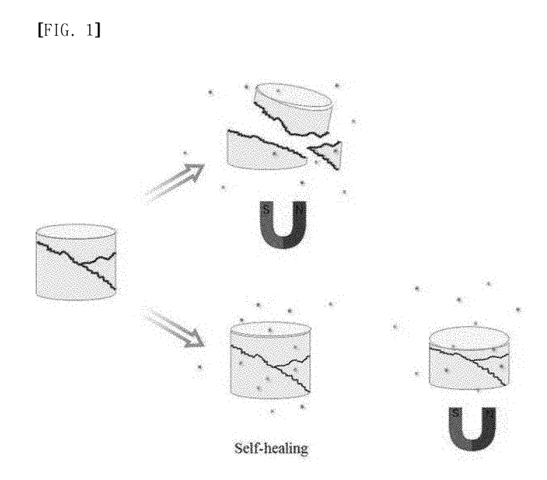

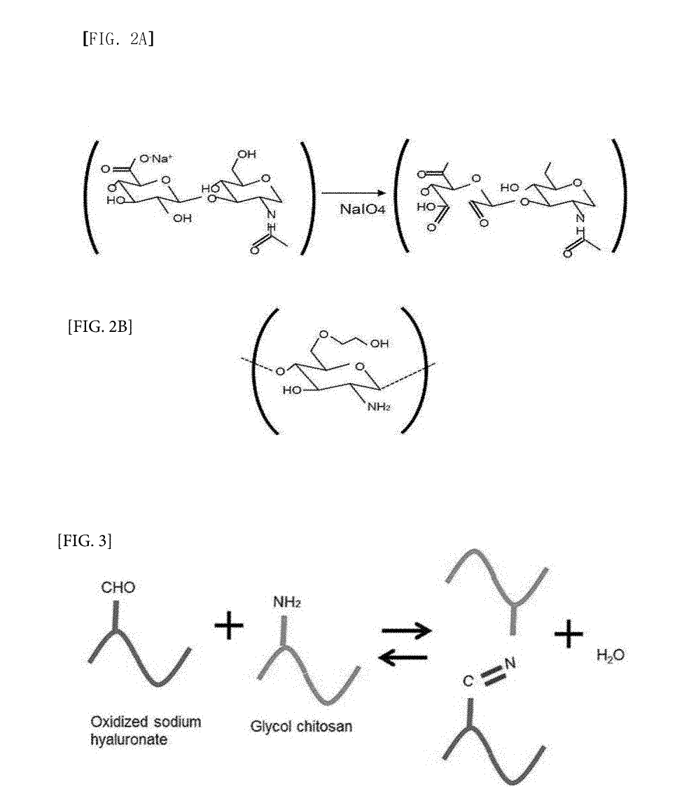

### DETAILED DESCRIPTION OF EXEMPLARY EMBODIMENTS

As used herein, the term “hydrogel” means a three-dimensional structure of a hydrophilic polymer retaining a sufficient amount of water. The hydrogel of the present invention means a glycol chitosan-based or hyaluronate-based hydrogel, and may be a chitosan-oxidized hyaluronate hydrogel or a hydrogel in which chitosan, oxidized hyaluronate, and iron oxide are bound to one another, according to a specific example. The hydrogel in which chitosan, oxidized hyaluronate, and iron oxide are bound to one another may be referred to as a ferrogel in the present invention.

As used here, the term “physiologically active substance” means a substance used for treatment, healing, prevention, diagnosis, or the like of a disease, and is not limited to specific substances or classification. Such physiologically active substances include organic synthetic compounds, extracts, proteins, peptides, nucleic acids, lipids, carbohydrates, steroids, extracellular matrix materials, cells, and the like.

The term “drug” as used herein may also be included in the physiologically active substances. In addition, various excipients used in the art, such as any diluent, release retardant, inert oil, binder, and the like may be optionally mixed.

As used herein, the term “physiologically active substance delivery vehicle” means a device capable of carrying or chemically binding a physiologically active substance and delivering the physiologically active substance into the living body.

As used herein, the term “support for inducing tissue regeneration” means a biocompatible support that induces tissue regeneration in such a way that a peptide having a function of inducing tissue regeneration adheres to the biocompatible support, and thus the interaction between a hydrogel and a cell is improved.

Hereinafter, the present invention will be described in more detail.

The present invention provides a hydrogel, comprising glycol chitosan and oxidized hyaluronate.

The hydrogel according to the present invention is characterized in that the hydrogel can be formed by including glycol chitosan without containing a crosslinking agent in hyaluronate. As a result, the hydrogel has no toxicity caused by a crosslinking agent, and thus has excellent stability in comparison with a hydrogel containing a crosslinking agent, in a case of being used as a delivery vehicle for delivering a substance into the living body. In addition, it has been experimentally identified that viscoelastic properties of the hydrogel of the present invention and its degradation resistance properties retained in a physiological environment make the hydrogel suitable for effective use in 3D printers, in vivo materials, or the like.

In the hydrogel of the present invention, an amino group of the glycol chitosan and an aldehyde group of the oxidized hyaluronate form an imine bond by the Schiff base reaction, so that a hydrogel can be formed. Accordingly, the present invention may provide a hydrogel (OHA/GC) characterized in that oxidized hyaluronate and glycol chitosan form an imine bond by the Schiff base reaction.

The oxidized hyaluronate means a substance in which the C2-C3 bond of hyaluronic acid is destroyed by using an oxidizing agent and an aldehyde group is present at the C2 and C3 positions; and degree of oxidation means the number of oxidized polymer repeating units per 100 polymer repeating units, which may be understood as mol %. For the oxidized hyaluronate of the present invention, any oxidized hyaluronate can be used as long as the oxidized hyaluronate is obtained by the oxidation of hyaluronic acid, and preferably a degree of oxidation thereof may be 10% to 80%, 20% to 70%, or 30% to 60%.

In a specific embodiment, a hydrogel was prepared using oxidized hyaluronate having a degree of oxidation of 50%, and an experiment was conducted.

As can be seen from the following examples, for the hydrogel according to the present invention, physical properties thereof can be regulated according to a content ratio of oxidized hyaluronate and glycol chitosan which constitute the hydrogel, or a degree of oxidation of oxidized hyaluronate.

A weight ratio of the glycol chitosan to the oxidized hyaluronate may be equal to or greater than 0.2:1 or 0.5 to 10:1. In a case where the content of the glycol chitosan is higher than that of the hyaluronate, the hydrogel itself may exhibit higher physical properties.

The hydrogel of the present invention may contain the glycol chitosan and the oxidized hyaluronate in an amount of 1.0 wt % or greater, preferably 1.0 wt % to 10.0 wt %, and more preferably 1.0 wt % to 5.0 wt %, with respect to the total weight of the hydrogel.

The hydrogel of the present invention may further comprise iron oxide.

In a case where iron oxide (Fe₃O₄) is further contained, the hydrogel can exhibit a further improved self-healing ability, and it is possible to prepare a magnetic hydrogel of which a self-healing ability can be regulated by a magnetic field. The iron oxide may be included in the hydrogel of the present invention in the form of nanoparticles. The iron oxide nanoparticles may be nanoparticles having a diameter of 10 to 30 nm.

The iron oxide may be contained in an amount of 1 wt % or greater preferably 3 wt % or greater, and more preferably 5 wt % or greater, with respect to the total weight of the hydrogel.

In an embodiment of the present invention, it has been experimentally identified that in a case where iron oxide is contained in an amount of 3 wt % or greater with respect to the total weight of the hydrogel, an excellent self-healing ability of 80% or higher is exhibited; and in a case where iron oxide is contained in an amount of 5 wt % or greater with respect to the total weight of the hydrogel, a remarkably excellent self-healing ability is exhibited.

A weight ratio of glycol chitosan to iron oxide nanoparticles in the hydrogel of the present invention may be 1:4.0 to 30, and preferably 1:4.0 to less than 15. In a case where the weight ratio satisfies the above range, the hydrogel can exhibit a better self-healing ability.

In addition, for the hydrogel of the present invention, a weight ratio of the total amount of polymers (sum of GC and OHA) in the hydrogel to iron oxide nanoparticles may be 1:0.5 to 1:10, or may be 1:1 to 1:7. The content of the iron oxide nanoparticles can be regulated by physical properties of the hydrogel depending on a content ratio of hyaluronate to chitosan in the hydrogel itself, or the like. For example, in a case where the content of GC in the hydrogel is higher than that of OHA, the hydrogel may exhibit higher physical properties, and thus more iron oxide may be contained.

In addition, in a case where glycol chitosan is contained in an amount of 1 wt % or greater with respect to the total weight of the hydrogel, the hydrogel can exhibit a better self-healing ability. In a case where the content of glycol chitosan is higher than that of hyaluronate, the hydrogel itself may exhibit higher physical properties. Therefore, in a case where a concentration of glycol chitosan is higher, more iron oxide nanoparticles may be contained to increase a self-healing ability of the hydrogel.

In addition, the present invention provides a physiologically active substance delivery vehicle, comprising the hydrogel. The physiologically active substance delivery vehicle can deliver a physiologically active substance as an active ingredient.

The delivery vehicle is a system capable of carrying or chemically binding a physiologically active substance and delivering the physiologically active substance into the living body, in which the delivery can be performed by causing the physiologically active substance to be carried on the hydrogel of the present invention and injecting the hydrogel into the living body.

Depending on the purpose, the physiologically active substance is allowed to be released constantly over a predetermined period at a predetermined site. In addition, in a case where a desired drug capable of regulating physiological activity is bound to or carried on the oxidized hyaluronate or glycol chitosan, and then a hydrogel is formed, the hydrogel may serve as a drug delivery vehicle. At this time, another physiologically active substance may be further carried on or bound to the hydrogel together, and the resulting hydrogel can be used.

Such an injectable regulated substance delivery vehicle is useful in a case where a drug with a low delivery rate to the patient's disease site is used, where a high-priced administered drug is excreted out of the body too quickly and lost, or where a drug with high side effects due to taking the same is used. That is, the injectable regulated substance delivery vehicle has advantages that a drug can be directly delivered to a diseased site, thereby maintaining a certain drug concentration at the area to be treated for a long time by regulating a releasing rate of the drug such that the drug is slowly released into the affected area, or that a drug can be locally delivered to an affected area by an injection method.

In the hydrogel of the present invention, a delivery rate of the physiologically active substance or the like can be regulated depending on the type and concentration of a drug chemically bound to the gel, the physical strength and chemical characteristics of the gel, the degradation rate of the gel, and the like.

As organic synthetic compounds which can be physically carried on or chemically bound to the hydrogel of the present invention and can be delivered into the living body, there are commonly used antibiotics, anti-cancer agents, anti-inflammatory analgesics, anti-viral agents, anti-bacterial agents, and the like.

As antibiotics, antibiotics selected from derivatives and mixtures of tetracycline, minocycline, doxycycline, ofloxacin, levofloxacin, ciprofloxacin, clarithromycin, erythromycin, cefaclor, cefotaxime, imipenem, penicillin, gentamycin, streptomycin, vancomycin, and the like can be exemplified.

As anti-cancer agents, anti-cancer agents selected from derivatives and mixtures of methotrexate, carboplatin, taxol, cisplatin, 5-fluorouracil, doxorubicin, etoposide, paclitaxel, camptothecin, cytosine arabinose, and the like can be exemplified.

As anti-inflammatory agents, anti-inflammatory agents selected from derivatives and mixtures of indomethacin, ibuprofen, ketoprofen, piroxicam, flurbiprofen, diclofenac, and the like can be exemplified.

As anti-viral agents, anti-viral agents selected from derivatives and mixtures of acyclovir, ribavirin, and the like can be exemplified.

As anti-bacterial agents, anti-bacterial agents selected from derivatives and mixtures of ketoconazole, itraconazole, fluconazole, amphotericin B, griseofulvin, and the like can be exemplified.

As proteins and peptides which can be carried on the hydrogel of the present invention and delivered into the living body, various physiologically active peptides such as hormones, cytokines, enzymes, antibodies, growth factors, transcriptional regulatory factors, blood factors, vaccines, structural proteins, ligand proteins, polysaccharides and receptors, cell surface antigens, and receptor antagonists, which are used for the purpose of treating or preventing a disease, and derivatives and analogues thereof can be exemplified.

Specifically, the following proteins and peptides can be exemplified: Liver growth hormones, growth hormone-releasing hormones, growth hormone-releasing peptides, interferons and interferon receptors (for example, interferon-alpha, -beta, and -gamma, water-soluble type I interferon receptor, and the like), granulocyte colony-stimulating factor (GCSF), granulocyte-macrophage colony-stimulating factor (GM-CSF), glucagon-like peptides (GLP-1 and the like), G-protein-coupled receptors, interleukins (for example, interleukin-1, -2, -3, -4, -5, -6, -7, -8, -9, and the like) and interleukin receptors (for example, IL-1 receptor, IL-4 receptor, and the like), enzymes (for example, glucocerebrosidase, iduronate-2-sulfatase, alpha-galactosidase-A, agalsidase alpha and beta, alpha-L-iduronidase, butyrylcholinesterase, chitinases, glutamate decarboxylase, imiglucerase, lipases, uricase, platelet-activating factor acetylhydrolase, neutral endopeptidase, myeloperoxidase, and the like), interleukin and cytokine binding proteins (for example, IL-18BP, TNF-binding proteins, and the like), macrophage activating factors, macrophage peptides, B cell factors, T cell factors, protein A, allergy suppressors, tumor necrosis factor (TNF) alpha suppressors, cell necrosis glycoproteins, immunotoxins, lymphotoxins, tumor necrosis factors, tumor suppressors, transforming growth factors, alpha-1 antitrypsin, albumin, alpha-lactalbumin, apolipoprotein-E, erythropoietin, highly-glycosylated erythropoietin, angiopoietin, hemoglobin, thrombin, thrombin receptor activating peptides, thrombomodulin, blood factor VII, blood factor VIIa, blood factor VIII, blood factor IX, blood factor XIII, plasminogen activators, fibrin-binding peptides, urokinase, streptokinase, hirudin, protein C, C-reactive protein, renin suppressors, collagenase suppressors, superoxide dismutase, leptin, platelet-derived growth factor, vascular endothelial growth factors, epidermal growth factor, angiostatin, angiotensin, bone morphogenic protein, bone morphogenesis promoting protein, calcitonin, insulin, atriopeptin, cartilage inducing factors, elcatonin, connective tissue activators, tissue factor pathway inhibitors, follicle stimulating hormone, luteinizing hormone, luteinizing hormone-releasing hormone, neurotrophines (for example, nerve growth factor, cilliary neurotrophic factor, axogenesis factor-1, brain-natriuretic peptide, glial derived neurotrophic factor, netrin, neurophil inhibitor factor, neurotrophic factors, neuturin, and the like), parathyroid hormone, relaxin, secretin, somatomedin, insulin-like growth factors, corticosteroids, glucagon, cholecystokinin, pancreatic polypeptides, gastrin-releasing peptides, corticotrophin-releasing factors, thyroid stimulating hormone, autotaxin, lactoferrin, myostatin, receptors (for example, TNFR(P75), TNFR(P55), IL-1 receptor, VEGF receptors, B cell activator receptor, and the like), receptor antagonists (for example, IL1-Ra and the like), cell surface antigens (for example, CD 2, 3, 4, 5, 7, 11a, 11b, 18, 19, 20, 23, 25, 33, 38, 40, 45, 69, and the like), monoclonal antibodies, polyclonal antibodies, antibody fragments (for example, scFv, Fab, Fab′, F(ab′)2, and Fd), virus-derived vaccine antigens and the like can be exemplified.

As nucleic acids which can be physically carried on or chemically bound to the hydrogel of the present invention and delivered into the living body, DNA, RNA, PNA, oligonucleotides, and the like can be exemplified.

As extracellular matrix materials which can be physically carried on or chemically bound to the hydrogel of the present invention and can be delivered into the living body, materials selected from the group consisting of collagen, fibronectin, gelatin, elastin, osteocalcin, fibrinogen, fibromodulin, tenascin, laminin, osteopontin, osteonectin, perlecan, versican, von Willebrand factor, and vitronectin, and the like can be exemplified.

As polysaccharide materials which can be physically carried on or chemically bound to the hydrogel of the present invention and can be delivered into the living body, heparin, heparan sulfate, keratan sulfate, dermatan sulfate, chondroitin sulfate, hyaluronate, and the like can be exemplified.

As cells which can be physically carried on the hydrogel of the present invention and delivered into the living body, fibroblasts, vascular endothelial cells, smooth muscle cells, neuronal cells, cartilage cells, bone cells, skin cells, Schwann cells, stem cells, and the like can be exemplified.

In addition, the hydrogel according to the present invention is excellent in self-healing ability as described above. Thus, the present invention provides a composition for three-dimensional bio-printing, comprising the hydrogel.

In addition, the present invention provides a method for preparing a hydrogel, comprising mixing glycol chitosan and oxidized hyaluronate.

The method for preparing a hydrogel may further comprise oxidizing hyaluronic acid to prepare oxidized hyaluronate.

The method for preparing a hydrogel may further comprise mixing iron oxide with the mixture of glycol chitosan and oxidized hyaluronate.

In a case where a hydrogel is prepared according to the preparation method of the present invention, due to the fact that no crosslinking agent is contained, it is possible to prepare a hydrogel which has an advantage of no toxicity and has a self-healing ability. In particular, the hydrogel has an advantage that the self-healing ability can be regulated by applying magnetism.

The details for the hydrogel as described above can be applied to the composition for three-dimensional bio-printing and the method for preparing a hydrogel, of the present invention.

Hereinafter, the present invention will be described in detail with reference to examples.

However, the following examples are intended only to more specifically describe the present invention. It will be apparent to those skilled in the art that in accordance with the gist of the present invention, the scope of the present invention is not limited by these examples.

### EXAMPLES

### <Preparation Example> Experimental Materials and Experimental Methods

### 1-1. Experimental Materials

Sodium hyaluronate (MW 1,000,000 g/mol) was purchased from Humedix Co., Ltd. Glycol chitosan (MW 50,000) was purchased from Wako Pure Chemical Industries, Ltd. Superparamagnetic iron oxide nanoparticles (Fe₃O₄, diameter: 20 nm) were purchased from US Research Nanomaterials, Inc. Formaldehyde was purchased from Junsei Chemical Co., Ltd., 2,4,6-trinitrobenzenesulfonic acid was purchased from Thermo Scientific, and Dulbecco's phosphate buffered saline (DPBS) was purchased from Gibco. Bovine serum albumin was purchased from Thermo Scientific.

### 1-2. Preparation of Oxidized Hyaluronate

1 g of hyaluronate (HA) was dissolved in 90 mL of distilled water. 0.2673 g of sodium periodate dissolved in 10 mL of distilled water was added to the HA solution, and mixed in the dark so that oxidation proceeded. After 24 hours of reaction, ethylene glycol was added to stop the oxidation. Then, the solution was purified for 3 days by dialysis with distilled water. Next, the solution was treated with activated charcoal, filtered through a 0.22 um filter, and lyophilized.

### 1-3. Chemical Analysis of Hydrogels

Fourier transform infrared spectroscopy (Nicolet iS50; Thermo Scientific) was used to identify the formation of imine bonds. The number of aldehyde groups in oxidized hyaluronate was measured using 2,4,6-nitrobenzene sulfonic acid (TNBS). Formaldehyde was used as a standard sample (0, 0.0813, 0.1625, 0.2438 mM). 0.5 mL of each sample was mixed with 0.25 mL of TNBS (0.01%). After incubation at 37° C., 0.25 mL of a sodium dodecyl sulfate solution (10%) and 0.125 mL of HCl (1 N) were added. Absorbance was measured at 335 nm using a spectrophotometer (SpectraMax M2; Molecular Devices).

### 1-4. Preparation and Characterization of Hydrogels

1 g of glycol chitosan was dissolved in 100 ml of distilled water. The same purification process (dialysis and lyophilization) described above was applied to the solution, so that glycol chitosan was prepared.

In order to prepare hydrogels, glycol chitosan (GC) and oxidized hyaluronate (OHA, degree of oxidation of 50%) were dissolved in DPBS at various mixing ratios ([GC]:[OHA]=0.5:1, 1:1, 2:1, 3:1, 5:1, and 7:1). The mixing ratio by volume was kept constant at [GC]:[OHA]=1:1, and the total concentration of polymers was 3 wt %. Hydrogel disks were fabricated (diameter: 11 mm; thickness: 1 mm).

In order to identify equilibrium swelling, the gels were immersed in DPBS for one day. The wet weight (Wₜ) and dry weight (W_{d}) of the gel disks were measured and the degree of swelling (Q) was calculated from the following equation:

Q(%)=100×(Wₜ−W_{d})/W_{d}

The elastic modulus of the gel disks was measured at 25° C. using a rotational viscometer (Gemini 150; Malvern Instruments) equipped with a parallel plate fixture.

### 1-5. Degradation Test

OHA/GC gel disks (diameter: 11 mm; thickness: 1 mm) were fabricated with the hydrogels and were immersed in DPBS for 21 days. The media were changed every other day. On days 1, 7, 14, and 21, the dry weight and elastic modulus of each gel disk were measured. The elastic modulus of the hydrogel was measured at 25° C. using a rotational viscometer (Gemini 150; Malvern Instruments) equipped with a parallel plate fixture.

### 1-6. Rheological Measurement

The viscoelastic properties of OHA/GC hydrogels were measured using a rotational viscometer (Gemini 150; Malvern) with a cone-and-plate fixture. The viscoelastic properties were measured in a frequency sweep mode and the temperature was kept at 37° C.

### 1-7. Preparation and Characterization of Self-Healing Hydrogels

Hydrogels were formed by mixing oxidized hyaluronate, glycol chitosan, and iron oxide nanoparticles while fixing the total polymer concentration ([polymer]=3 wt %) and varying a concentration of iron oxide nanoparticles (5 wt %, 10 wt %, 15 wt %), and viscoelastic properties thereof were identified. Respective hydrogels were formed while fixing an iron oxide concentration to 5 wt % and varying the total polymer concentration (1.8 wt %, 2.4 wt %, 3.0 wt %), and viscoelastic properties thereof were identified.

Specifically, in a case where a hydrogel with a mass ratio of glycol chitosan to oxidized hyaluronate being 5:1 is used and the content of iron oxide nanoparticles is 0 wt %, 1 wt %, 3 wt %, 5 wt %, 10 wt %, or 15 wt % with respect to the total hydrogel, the self-healing behavior of the hydrogel was identified. In addition, when the total polymer content (total content of [GC] and [OHA], [GC]:[OHA]=5:1 (mass ratio)) in a hydrogel is 1.8 wt %, 2.4 wt %, or 3.5 wt % in a case where the content of the iron oxide nanoparticles is 0 wt % and 15 wt %, the self-healing behavior of the hydrogel was identified.

The self-healing behavior depending on the concentration of iron oxide nanoparticles (Fe₃O₄) was identified using a rotational rheometer (25° C., equipped with a cone-and-plate fixture). In order to measure the gel breaking strain, an amplitude sweep was carried out in the range of 1% to 300% strain. 400% strain was applied for 2 minutes to break the gels, and the strain was removed. Next, after a certain time, a self-healing ability was evaluated as a degree of recovery of physical properties. That is, the recovery of elastic modulus was monitored using a rotational viscometer, and the self-healing behavior of hydrogels (ferrogels) prepared with various concentrations of iron oxide nanoparticles and polymers was investigated. Ferrogel disks were fabricated (diameter: 11 mm; thickness: 1 mm) and cut into 4 pieces. Then, the gel pieces were gathered by applying the magnetic field, and the elastic moduli of the original gel disk and the self-healed gel disk were measured.

### 1-8. Macroscopic Observation of Self-Healing Performance

The GC/OHA/Fe₃O₄ hydrogels thus prepared were fabricated into disk shapes (diameter: 11 mm; thickness: 3 mm). The gel disks were assembled together, and a magnetic field was applied thereto. Then, after 10 minutes, the self-healing behavior thereof was monitored.

### 1-9. In Vitro Release Test of BSA

Bovine serum albumin (BSA) was added to a glycol chitosan solution in DPBS, and GC/OHA/Fe₃O₄ ferrogels were fabricated as described above. The ferrogels were cut into disk shapes (diameter: 11 mm; thickness: 1 mm) and immersed in DPBS (1 mL). The ferrogel disks were stimulated by applying the magnetic field for 3 minutes every hour, and the concentration of BSA released from the disks was measured at 562 nm using a spectrophotometer.

### <Experimental Results>

### 2-1. Preparation of GC/OHA Hydrogels

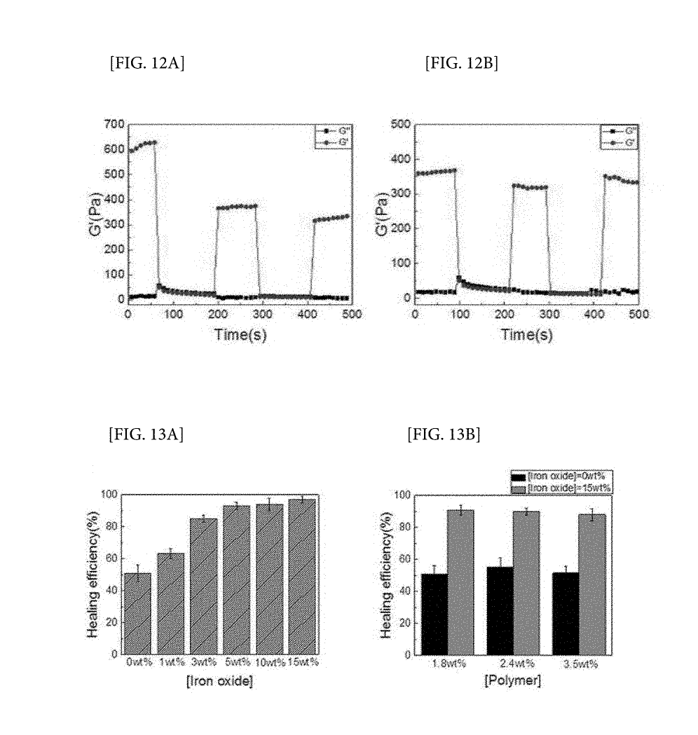

In order to identify whether hydrogels can be prepared by mixing glycol chitosan and oxidized hyaluronate, hydrogels were prepared with varying mixing ratios of the two components, and a self-healing ability thereof was identified. In addition, hydrogels were prepared using the glycol chitosan (GC) and oxidized hyaluronate (OHA; degree of oxidation of 50%) while fixing the total polymer concentration ([polymer]=3 wt %) and varying a mass ratio of glycol chitosan to oxidized hyaluronate (0.5:1, 1:1, 2:1, 3:1, 5:1, or 7:1). It was identified that hydrogels were formed in all experimental groups. However, a self-healing ability was hardly exhibited (FIG. 12(a)).

In addition, the new cross-linking in hydrogels was identified by FT-IR.

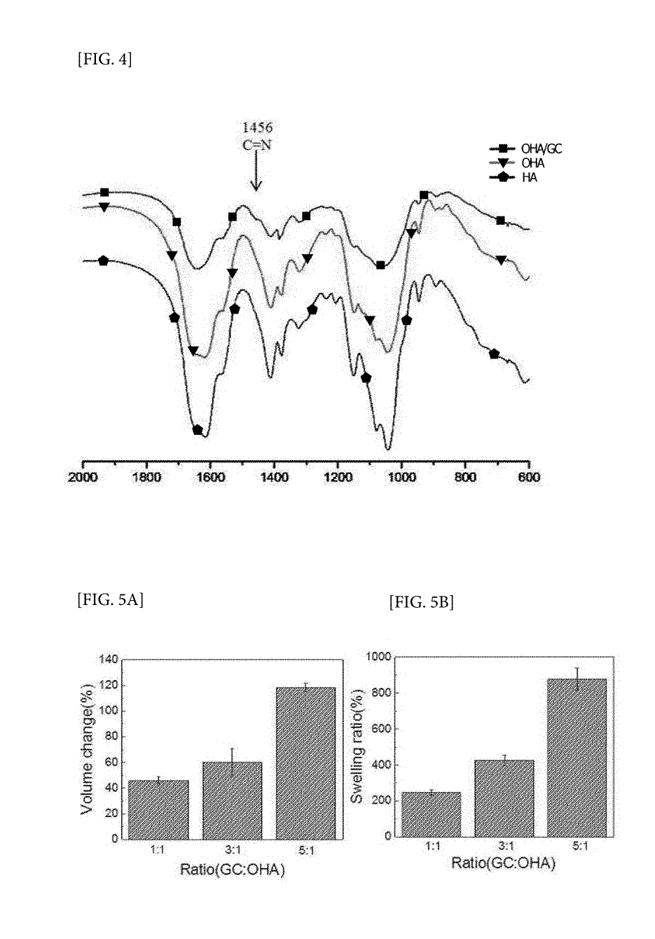

As shown in FIG. 4, imine bonds were found at 1456 cm⁻¹. In order to identify the number of aldehyde groups in oxidized hyaluronate, TNBS assay was carried out. Because aldehyde groups do not react with TNBS, an excess amount of TBC was present and the remaining TBC content was determined. Formaldehyde was used as a standard reagent. The actual degree of oxidation for 50-OHA was 57.5±6.4 (Table 1).

TABLE 1

| | | Theoretical degree | Actual degree |
|---|---|---|---|
| Sample | NaIO₄ (mg)/1 g HA | of oxidization (%) | of oxidization (%) |
| 50-OHA | 267.35 | 50 | 57.54 ± 6.4 |

### 2-2. Swelling Behavior of GC/OHA Hydrogels

In order to optimize the GC/OHA mixing ratio, the swelling ratio and volume change of gels prepared with various GC/OHA ratios ([GC]:[OHA]=1:1, 3:1, 5:1) were investigated.

As shown in FIG. 5, the swelling ratio increased as the GC content increased, and the volume also increased as the GC content increased. Thus, hydrogels prepared with [GC]:[OHA]=5:1 were chosen and used for further experiments.

### 2-3. Rheological Characterization of GC/OHA Hydrogels

The moduli of hydrogels with different contents of GC ([GC]:[OHA]=1:1, 3:1, 5:1) were investigated.

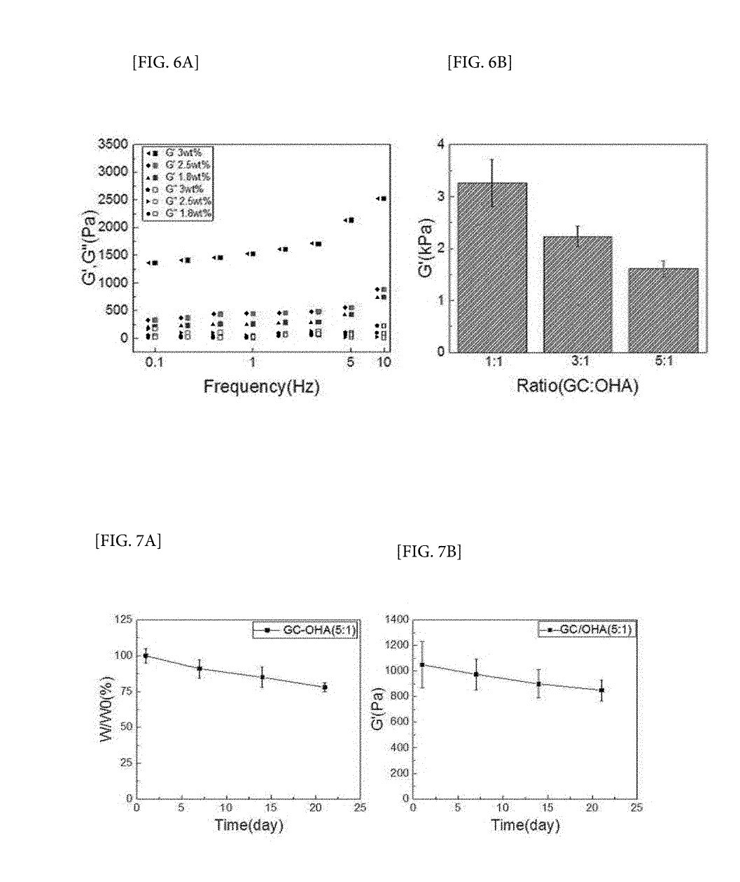

As shown in FIG. 6, the mechanical properties of GC/OHA gels increased as the GC content decreased. In addition, the elastic moduli of hydrogels decreased as the polymer concentration decreased.

### 2-4. Identification of Degradation of GC/OHA Hydrogels

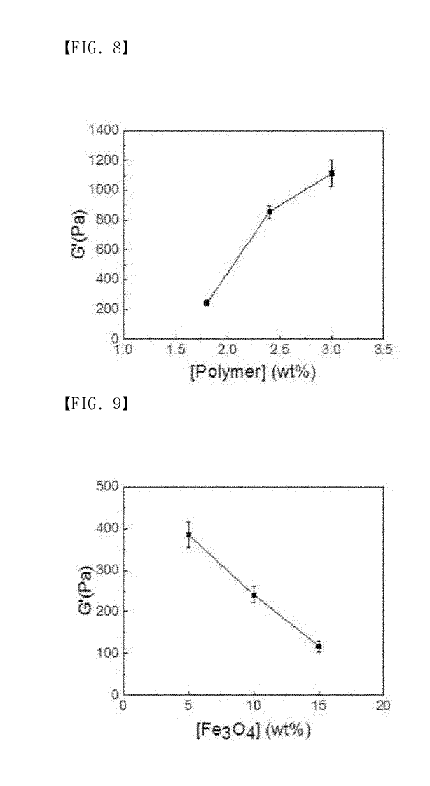

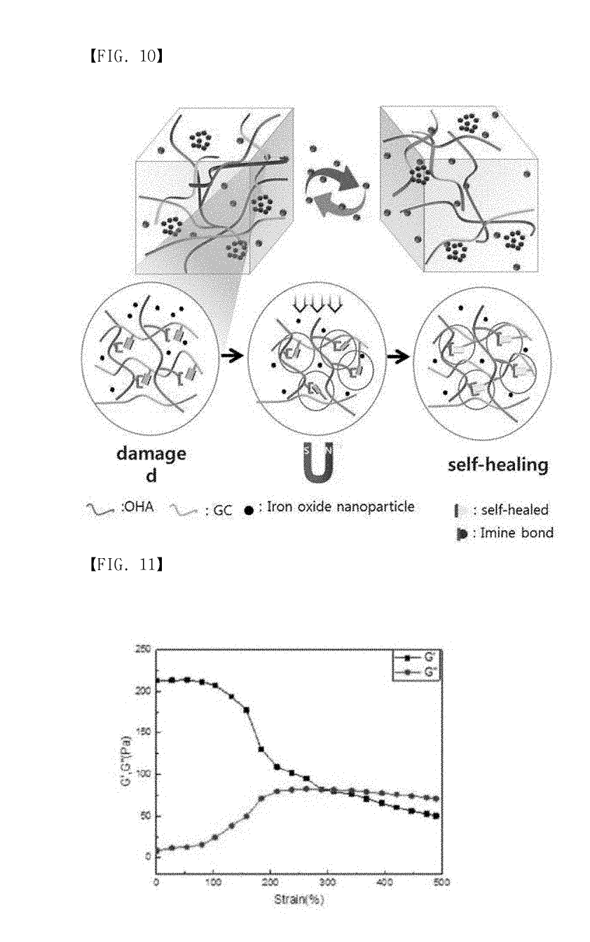

As shown in FIG. 7, disks prepared from GC/50-OHA gels maintained more than 80% of their original weight in DPBS. In addition, gels maintained their elastic modulus in comparison with the original elastic modulus. From these results, it was identified that the hydrogels prepared according to the present invention are not degraded in a physiological environment and can maintain a gel form. Thus, it can be seen that GC/50-OHA hydrogels are suitable for various applications including three-dimensional printing and drug delivery systems.

2-5. Identification of Viscoelastic Properties of GC/OHA/Fe₃O₄ Hydrogels (Ferrogels)

GC/OHA hydrogels were prepared in the presence of iron oxide nanoparticles, and it was checked whether reversible gelation can occur in such hydrogels.

As shown in FIGS. 8 and 9, the storage shear moduli of the GC/OHA/Fe₃O₄ hydrogels ([Fe₃O₄]=5 wt %) changed depending on polymer concentration, and addition of iron oxide nanoparticles caused a decrease in storage moduli of the gels.

In addition, it was identified that the GC/OHA/Fe₃O₄ hydrogels show a self-healing behavior due to magnetism. The hydrogels containing no iron oxide nanoparticles do not show this self-healing behavior, which confirms that the iron oxide nanoparticles play an important role in making hydrogels reversible.

2-6. Self-Healing Efficiency of GC/OHA/Fe₃O₄ Hydrogels

GC/OHA/Fe₃O₄ hydrogels showed a self-healing behavior, which was analyzed by a rotational rheometer.

The minimum strain that breaks a GC/OHA/Fe₃O₄ hydrogel was determined by an amplitude sweep and was found to be 288% (FIG. 11). The GC/OHA/Fe₃O₄ hydrogels showed a self-healing behavior under 400% strain that was applied every 2 minutes (FIG. 12(b); FIG. 12(a) illustrates GC/OHA hydrogels containing no iron oxide as a comparison group).

The initial modulus before gel breaking and the modulus after self-healing were measured and this was repeated twice. When the concentration of iron oxide nanoparticles reached 5 wt %, the GC/OHA/Fe₃O₄ hydrogels showed a self-healing behavior (FIG. 13(a)). Changes in self-healing ability were checked in a case where the total polymer concentration is 1.8 wt %, 2.4 wt %, or 3.5 wt % when the concentration of the iron oxide nanoparticles contained in the hydrogels is fixed to 0 wt % and 15 wt %, and a mass ratio of glycol chitosan to hyaluronic acid is 5:1. As a result, polymer concentration did not significantly affect self-healing ability (FIG. 13(b)).

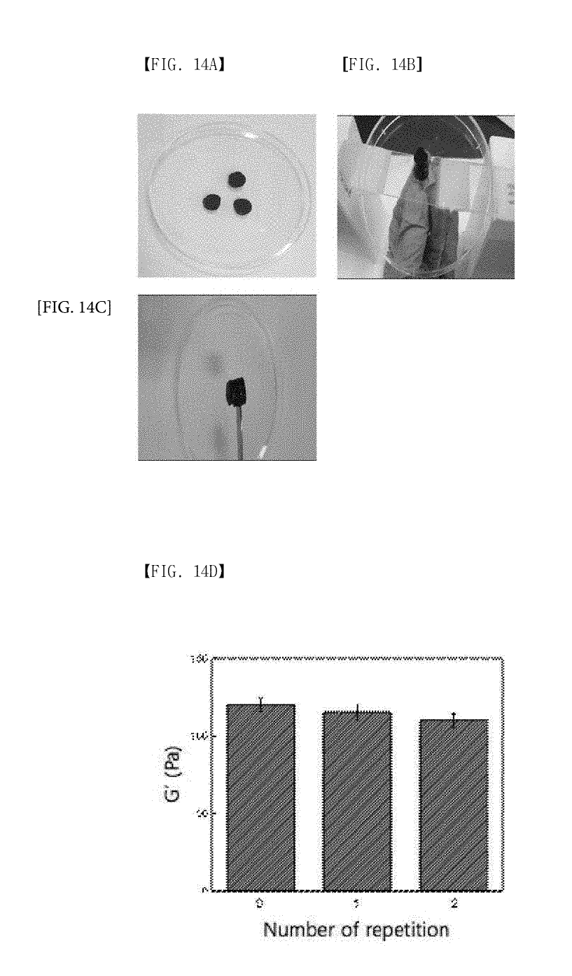

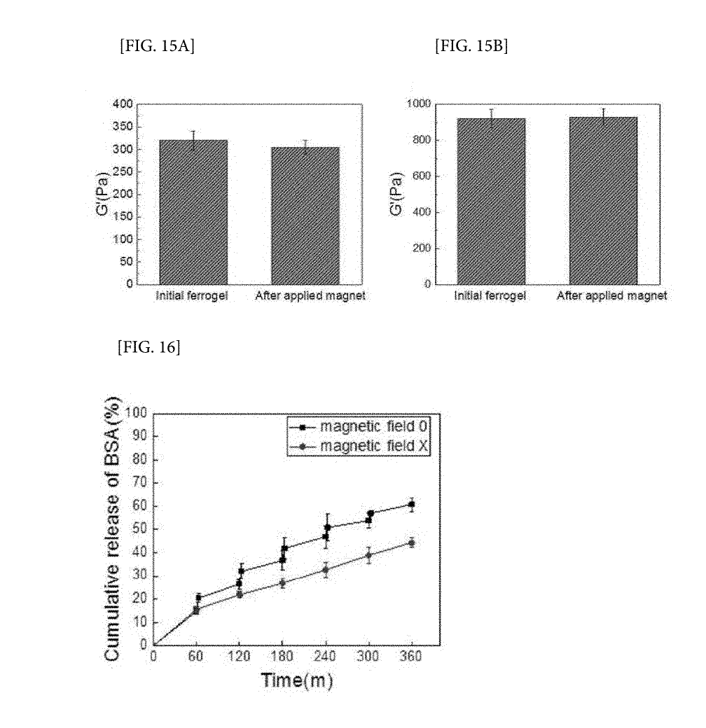

The self-healing behavior of the GC/OHA/Fe₃O₄ hydrogel disks was also visually checked (FIG. 14). As shown in FIG. 14(a), it was found that in a case where the magnetic field is applied to GC/OHA/Fe₃O₄ hydrogel disks (diameter: 11 mm; thickness: 3 mm), the disks are self-healed after 10 minutes. As shown in FIG. 15, it was found that as a result of making an elastic modulus comparison between initial gel disks and gel disks after application of the magnetic field, there are no significant changes. In addition, a procedure, in which the GC/OHA/Fe₃O₄ hydrogel disk (diameter: 10 mm, [GC]:[OHA]=5:1, 15 wt % of iron oxide nanoparticles) is cut into 4 pieces, the magnetic field is applied so that the gel is self-healed, and then physical properties of the gel are measured, was repeated to identify changes in physical properties of the magnetic hydrogel after self-healing. As shown in FIG. 14B, there was a slight decrease in physical properties due to repeated cutting. However, it was identified that the physical properties of the original magnetic hydrogel are well maintained. Therefore, it can be seen from the above results that the GC/OHA/Fe₃O₄ hydrogels of the present invention have a self-healing ability.

2-7. Identification of BSA-Releasing Ability from GC/OHA/Fe₃O₄ Hydrogels (Ferrogels)

In order to identify whether the magnetic hydrogel of the present invention having a self-healing ability can be used as a drug delivery vehicle, a drug release behavior thereof was checked.

A GC/OHA/Fe₃O₄ hydrogel was prepared by mixing 2.1 wt % of total polymers ([GC]:[OHA]=5:1 (mass ratio)), 15 wt % of iron oxide nanoparticles, and 0.06 wt % of bovine serum albumin (BSA) which is a modeling drug, and then GC/OHA/Fe₃O₄ hydrogel disks (diameter: 11 mm; thickness: 1 mm) were prepared therefrom. The disks were immersed in a DPBS solution, and then a magnetic field (0.3 T) was applied at 25° C. to stimulate the disks. In addition, a group not stimulated with the magnetic field was set as a control group. The magnetic field was applied to the gel every 3 minutes per hour.

As shown in FIG. 16, in a case of control disks without the magnetic field, the cumulative release of BSA reached 30% within 6 hours. However, it was identified that in a state of being treated with the magnetic field, the cumulative release of BSA reached 60% within 6 hours. From this fact, it can be seen that in a case where the magnetic field is applied to the GC/OHA/Fe₃O₄ hydrogels of the present invention, release of a drug from the gels can be regulated.

What is claimed is:

1. A hydrogel, comprising:
glycol chitosan; and
oxidized hyaluronate.

2. The hydrogel according to claim 1,
wherein a degree of oxidation of the oxidized hyaluronate is 10% to 80%.

3. The hydrogel according to claim 1, further comprising:
iron oxide.

4. The hydrogel according to claim 3,
wherein the iron oxide is contained in an amount of greater than 1 wt % with respect to the total weight of the hydrogel.

5. The hydrogel according to claim 3,
wherein the hydrogel has a self-healing ability.

6. The hydrogel according to claim 1,
wherein the total weight of the glycol chitosan and the oxidized hyaluronate is contained in an amount of 1.0 wt % to 5.0 wt % with respect to the total weight of the hydrogel.

7. A physiologically active substance delivery vehicle with a self-healing ability, comprising:
the hydrogel according to claim 1.

8. The physiologically active substance delivery vehicle according to claim 7,
wherein the physiologically active substance is selected from the group consisting of antibiotics, anti-cancer agents, analgesics, anti-inflammatory agents, anti-viral agents, anti-bacterial agents, proteins, peptides, nucleic acids, polysaccharides, lipids, carbohydrates, steroids, extracellular matrix materials, and cells.

9. The physiologically active substance delivery vehicle according to claim 8,
wherein the protein is selected from the group consisting of hormones, cytokines, enzymes, antibodies, growth factors, transcriptional regulatory factors, blood factors, vaccines, structural proteins, ligand proteins and receptors, cell surface antigens, and receptor antagonists.

10. The physiologically active substance delivery vehicle according to claim 8,
wherein the nucleic acid is selected from the group consisting of oligonucleotides, DNA, RNA, and PNA.

11. A method for preparing a self-healing hydrogel, comprising:
mixing glycol chitosan and oxidized hyaluronate; and
mixing iron oxide with the mixture.

12. The method according to claim 11,
wherein the glycol chitosan and the oxidized hyaluronate are mixed in a weight ratio of equal to or greater than 0.2:1.

13. The method according to claim 11,
wherein the iron oxide is mixed so that a content of the iron oxide is 1 wt % or greater with respect to the total weight of the hydrogel.

14. The method according to claim 12,
wherein the glycol chitosan and the oxidized hyaluronate are mixed so that the total sum of the glycol chitosan and the oxidized hyaluronate is 1.0 wt % to 10.0 wt % with respect to the total weight of the hydrogel.
